(19) United States
(12) Patent Application Publication
(10) Pub. No.: US 2006/0129030 A1
(43) Pub. Date: Jun. 15, 2006

(54) Ureteroscope with a distal beak

(75) Inventor: Joachim Dehmel, Hamburg (DE)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, Hamburg (DE)

(21) Appl. No.: 11/263,407

(22) Filed: Oct. 31, 2005

(30) Foreign Application Priority Data
Dec. 9, 2004 (DE) 102004059255.1-35

(51) Int. Cl. A61B 1/00; A61B 1/307

(52) U.S. Cl. 600/129; 600/135

(57) ABSTRACT

A ureteroscope (1) fitted with a stem (2) which exhibits at least at its distal end zone a substantially oval cross-section and which comprises a rounded beak (6) projecting at the distal end of the cross-section in the direction of the longer transverse axis (7), is characterized in that the beak (6) is asymmetrical to the transverse axis (7) in a manner that its center line (8) deviates increasingly from the transverse axis (7) as the radius increases.

Ureteroscopes of this kind are known as ureteroscopes with Mersier tips. The beak is used to more easily insert the stem from the bladder through the ostium into the ureter.

The ostium is defined as the ureter's entry zone. A resilient flap acting as check valve and sealing the ureter is situated at the ostium. When urine flows from the ureter into the bladder, the flap shall open, while it closes when there is excess urine pressure in the bladder to prevent the urine from backflowing into the ureter. As a result both renal overpressure and infections migrating through the ureter into the kidney are precluded.

Overcoming the ostium flap is always a problem for the surgeon when inserting ureteroscopes. The flap is easily injured, for instance by being squeezed. As a result the ostium flap no longer may reliably close, entailing the danger that any bladder infection might immediately give rise to infection of the renal pelvis.

The known Mersier tip of this kind contributes to keep the ostium flap free of injury. The beak allows seizing the flap underneath and to rotate it about its longitudinal axis by rotating the ureteroscope, without squeezing this flap.

However the known design of this kind still leaves a residual danger of injury. Known Mersier tips of the above kind comprise a beak symmetrical to the longer transverse axis of the stem cross-section, and, upon penetration, this beak then will rest disadvantageously underneath the sealing flap. Therefore the flap still may be squeezed.

The objective of the present invention is to reduce the danger of ostium flap injury relative to the known above cited kind.

In the present invention, the cross-section of the beak is asymmetrical to the transverse axis, the asymmetry increasing toward the outside. The beak shape so attained allows the flap to be seized underneath in a much simpler and effective manner, and this flap may be reliably opened in an injury-free manner by rotating the stem.

The present invention is shown illustratively and schematically in the appended drawings.

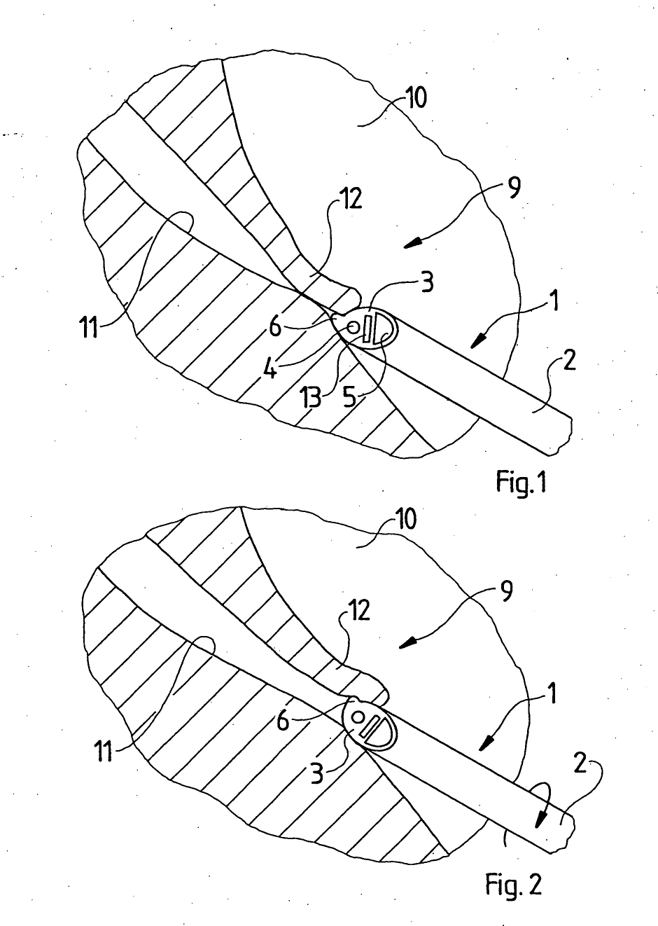

FIGS. 1-3 show an ostium cross-section when the ureteroscope of the invention is in three consecutive insertion phases, and

FIG. 4 is an enlarged front view of the ureteroscope.

The ureteroscope is shown in FIGS. 1-4 only by the distal end zone of its stem 2. An optics 4, an operational duct 5 and a light guide 13 issue into the distal end surface 3.

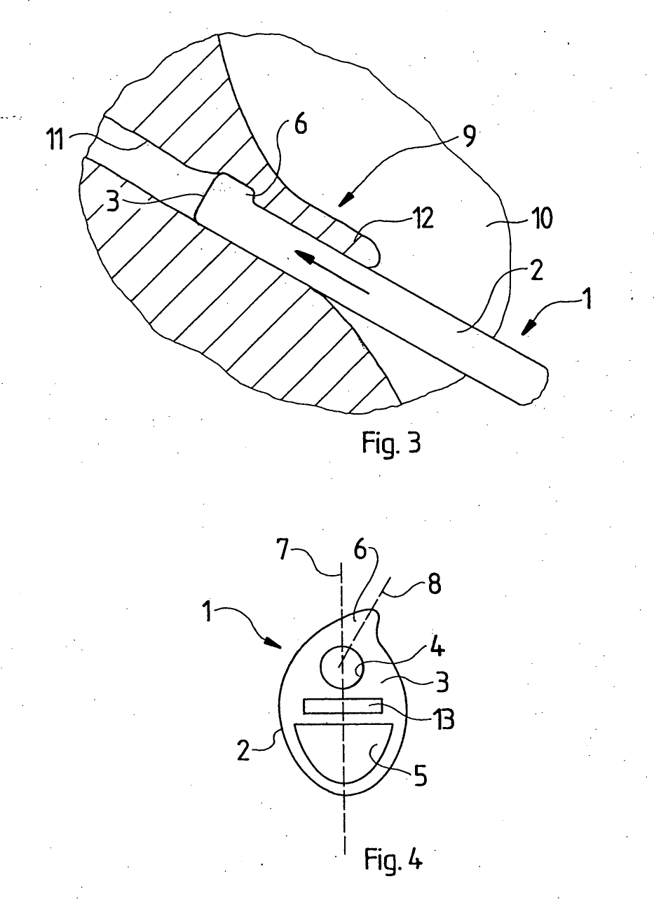

As shown by the sideview of FIG. 3, a well rounded beak 6 is mounted at the distal end of the stem 2 and laterally projects beyond the otherwise oval cross-section of the stem substantially in the direction of the longer transverse axis 7 (FIG. 4) of the cross-section.

As shown by FIG. 4 in front view of the end surface 3, the beak is asymmetrical in a way that it increasingly deviates by its center line 8 from the transverse axis 7 as the radius increases. Accordingly, it is slightly bent laterally, increasingly more with outward distance. The result is the shape of the beak 6 shown in FIGS. 1, 2 and 4. The center line 8 may be straight, as shown, or it also may be bent and run obliquely to the transverse axis 7.

FIGS. 1-3 show the procedure by which the ureteroscope is inserted in three consecutive steps.

In this regard, FIGS. 1-3 show the cross-section of the region of the ostium 9 of a human bladder 10, namely that region of a ureter 11 leading to the omitted kidney, which will terminate into the bladder 10. As shown by FIGS. 1-3, the ostium comprises a flap 12 which in its function as a check valve opening toward the bladder, resiliently seals the ureter 11.

The ureteroscope 1 of the invention is inserted into the ureter 11 in the following manner. First, in a manner not shown, the ureteroscope 1 is inserted into the bladder 10 and moved into position as shown in FIG. 1. At that position, the stem end is made to rest by its beak 6 against the edge of the flap 12 (as shown in FIG. 1), and now, by rotating the stem 2 in the direction of the arrow of FIG. 2, the flap 12 may be pried up and the stem tip can then be moved underneath the flap as far as into the entry to the ureter 11. Thereupon the ureteroscope 1 may be advanced in the direction of the arrow of FIG. 3 into the ureter 11.

As shown by FIGS. 1-3, the flap 12 is handled most gently in the above described procedure. Initially (FIGS. 1, 2) the flap is lifted very carefully, in the absence of any danger of being squeezed, and then, it will be pushed and kept aside until the position of FIG. 3 has been reached.

1. A ureteroscope (1) fitted with a stem (2) which exhibits at least at its distal end zone a substantially oval cross-section and which comprises a rounded beak (6) projecting at the distal end of the cross-section in the direction of the longer transverse axis (7),
characterized in that the beak (6) is asymmetrical to the transverse axis (7) in a manner that its center line (8) deviates increasingly from the transverse axis (7) as the radius increases.
